(19) United States
(12) Patent Application Publication
(10) Pub. No.: US 2015/0087679 A1
(43) Pub. Date: Mar. 26, 2015

(54) NUTRITIONAL SLEEP SUPPLEMENT

(71) Applicants: Hany HELMI, Statesboro, GA (US); Andrew PANDYA, Savannah, GA (US)

(72) Inventors: Hany HELMI, Statesboro, GA (US); Andrew PANDYA, Savannah, GA (US)

(21) Appl. No.: 14/493,228

(22) Filed: Sep. 22, 2014

Related U.S. Application Data
(60) Provisional application No. 61/880,411, filed on Sep. 20, 2013.

(51) Int. Cl. A61K 31/455; A61K 31/19; A61K 31/194; A23L 1/29; A61K 31/198; A61K 31/4045; A61K 31/4415; A61K 31/405; A61K 31/197

(52) U.S. Cl. CPC A61K 31/455; A61K 31/405; A61K 31/19; A61K 31/194; A61K 31/197; A61K 31/198; A61K 31/4045; A61K 31/4415; A23L 1/296; A23V 2002/00
USPC 514/345

(57) ABSTRACT

Embodiments of the present invention relate to a nutritional sleep supplement having a balanced ratio of nutritional elements to facilitate a healthy lifestyle. In one embodiment, a nutritional sleep supplement has a set of active ingredients, the active ingredients comprising: between 50 mg to 600 mg of 5-Hydroxytryptophan; between 125 mg to 1500 mg of calcium lactate; between 50 mg to 600 mg of citric acid; between 125 mg to 1500 mg of gamma-Aminobutyric acid; between 50 mg to 600 mg of L-theanine; between 25 mg to 300 mg of L-tryptophan; between 45 mg to 540 mg of magnesium glycinate; between 1.25 mg to 15 mg of melatonin; between 8.75 mg to 105 mg of niacin; between 7.5 mg to 90 mg of potassium sorbate; and between 10 mg to 100 mg of pyridoxine.

### CROSS-REFERENCE TO RELATED APPLICATIONS

This application claims priority to U.S. Provisional Patent Application Ser. No. 61/880,411, filed Sep. 20, 2013, entitled “Nutritional Sleep Supplement,” the disclosure of which is incorporated by reference herein in its entirety.

### BACKGROUND OF THE INVENTION

1. Field of the Invention

Embodiments of the present invention generally relate to a nutritional sleep supplement. More specifically, embodiments of the present invention relate to a nutritional sleep supplement having a balanced ratio of nutritional elements to facilitate a healthy lifestyle.

2. Description of the Related Art

There are countless nutritional supplements, or nutraceuticals, in the marketplace; each one claiming to be better than all others. Most of these nutritional supplements are nothing more than a multivitamin tablet, providing the consuming individual a proper balance of daily recommended minerals and vitamins. A proper diet, and nothing more, could achieve the same results as many of the nutritional supplements on the market.

Certain nutritional supplements are more specialized, and thus, are generally not easily substituted with a traditional healthy diet. For example, nutritional supplements for significant muscle growth, erectile dysfunction, etc., often comprise combinations of ingredients that may not be readily available from ordinary foods. As such, these nutritional supplements often provide benefits to even the most healthy individuals.

Other nutritional supplements, which claim to provide certain benefits may not be healthy or beneficial in the slightest. For example, several “sleep formulas” which hold themselves out as having no negative side effects, are often later found criticized in public for being the cause of headaches, pain or worse. In particular, some of these sleep formulas have so much alcohol or chemically-known equivalent, that when consumed in just small doses, can cause dependency issues.

As such, there is a need for a nutritional sleep supplement having a balanced ratio of nutritional elements to facilitate a healthy lifestyle.

### SUMMARY

Embodiments of the present invention generally relate to a nutritional sleep supplement. More specifically, embodiments of the present invention relate to a nutritional sleep supplement having a balanced ratio of nutritional elements to facilitate a healthy lifestyle.

In one embodiment of the present invention, a nutritional sleep supplement has a set of active ingredients, the active ingredients comprising: between about 50 mg to about 600 mg of 5-Hydroxytryptophan; between about 125 mg to about 1500 mg of calcium lactate; between about 50 mg to about 600 mg of citric acid; between about 125 mg to about 1500 mg of gamma-Aminobutyric acid; between about 50 mg to about 600 mg of L-theanine; between about 25 mg to about 300 mg of L-tryptophan; between about 45 mg to about 540 mg of magnesium glycinate; between about 1.25 mg to about 15 mg of melatonin; between about 8.75 mg to about 105 mg of niacin; between about 7.5 mg to about 90 mg of potassium sorbate; and between about 10 mg to about 100 mg of pyridoxine.

In another embodiment, a nutritional sleep supplement has a set of inactive ingredients, the inactive ingredients comprising: between about 47.5 mg to about 570 mg of sucralose; between about 7.5 mg to about 90 mg of sodium benzoate; and between about 0.001 ml to about 180 ml of water.

### DETAILED DESCRIPTION

It is understood that the embodiments of the present invention are not limited to the particular methodologies, protocols, solvents and reagents, and the like, described herein as they may vary. It is also to be understood the terminology used herein is used for the purpose of describing particular embodiments only and not intended to limit the scope of the present invention. It must also be noted that as used herein, the singular form “a,” “an” and “the” include the plural reference unless the context clearly dictates otherwise. Thus, for example, a reference to “a vitamin” is a reference to one or more vitamins and includes equivalents thereof known to those skilled in the art and so forth.

Unless defined otherwise, all technical and scientific terms used herein have the same meaning as commonly understood by one of ordinary skilled in the art to which this invention belongs. Exemplary methods, devices, elements, ingredients and materials are described, although any methods and materials similar or equivalent to those described herein could be used in the practice or testing of the present invention. All references cited herein are incorporated by reference herein in their entirety.

In accordance with embodiments of the present invention, the nutritional sleep supplement (hereinafter “supplement”) comprises a set of active ingredients and a set of inactive ingredients. The active ingredients are those which provide a targeted health-related or sleep-related function to the user upon consumption, and the inactive ingredients are those which complement the active ingredients with flavor, consistency, capsulation, or non-targeted health-related function.

In one embodiment of the present invention, the supplement may comprise a set of active ingredients, including 5-HTP, calcium lactate, citric acid, GABA, L-theanine, L-tryptophan, magnesium glycinate, melatonin, niacin, potassium sorbate, pyridoxine, sucralose and/or sodium benzoate. In another embodiment, the supplement may comprise a set of inactive ingredients, including flavorants, sugars or sweeteners, and water.

In terms of commercially prepared volumes, a supplement may comprise between about 500 to about 6,000 mg of active ingredients. In one particular embodiment, the supplement comprises about 2,045 mg of active ingredients. For purposes of the disclosure herein, the percentages of ingredients shown are generally provided in light of active ingredients by volume as any number of inactive ingredients may be added without deviating from embodiments of the present invention.

5-HTP (5-Hydroxytryptophan), also known as oxitriptan, is a naturally occurring amino acid and chemical precursor as well as a metabolic intermediate in the biosynthesis of the neurotransmitters serotonin and melatonin from tryptophan. It is commonly used as an antidepressant, appetite suppressant and sleep aid. In many embodiments of the present invention, a supplement comprises between about 2.44% to about 29.34%, or between about 50 mg to about 600 mg of 5-HTP. In one particular embodiment, the supplement comprises about 9.78%, or about 200 mg of 5-HTP.

Calcium lactate is most commonly used as an antacid and to treat calcium deficiencies. It is also used to assist in tooth decay and can be found in many over-the-counter mouth washes. In many embodiments of the present invention, a supplement comprises between about 6.11% to about 73.35%, or between about 125 mg to about 1500 mg of calcium lactate. In one particular embodiment, the supplement comprises about 24.45%, or about 500 mg of calcium lactate.

Citric acid is a weak organic acid. It is often added to foods as an emulsifying agent to keep fats from separating. It is also used in food products for its sour flavor or for when a dry ingredient is needed, but and substantial additional flavors are unwanted. In many embodiments of the present invention, a supplement comprises between about 2.44% to about 29.34%, or between about 50 mg to about 600 mg of citric acid. In one particular embodiment, the supplement comprises about 9.78%, or about 200 mg of citric acid.

GABA, or gamma-Aminobutyric acid, is the chief inhibitory neurotransmitter in the mammalian central nervous system. In humans, it is also directly responsible for the regulation of muscle tone. It is believed that GABA has a calming effect in humans as well. In many embodiments of the present invention, a supplement comprises between about 6.11% to about 73.35%, or between about 125 mg to about 1500 mg of GABA. In one particular embodiment, the supplement comprises about 36.67%, or about 750 mg of GABA.

L-theanine is an amino acid and a glutamic acid often found in tea and in certain mushrooms. While there are no statistically certain effects found with L-theanine, it is believed to have shown increased alpha wave generation and lowered anxiety; both of which lead to benefits to sleep quality, particularly in patients who have ADHD. In many embodiments of the present invention, a supplement comprises between about 2.44% to about 29.34%, or between about 50 mg to about 600 mg of L-theanine. In one particular embodiment, the supplement comprises about 9.78%, or about 200 mg of L-theanine.

L-tryptophan is an amino acid, a protein building block that can be found in many plant and animal proteins. L-tryptophan is called an “essential” amino acid because the body cannot naturally product it, and it must be acquired from food. L-tryptophan is used for insomnia, sleep apnea, depression, anxiety, facial pain, a severe form of premenstrual syndrome called premenstrual dysphoric disorder (PMDD), smoking cessation, grinding teeth during sleep (bruxism), attention deficit-hyperactivity disorder (ADHD), Tourette's syndrome, and to improve athletic performance. In many embodiments of the present invention, a supplement comprises between about 1.22% to about 14.67%, or between about 25 mg to about 300 mg of L-tryptophan. In one particular embodiment, the supplement comprises about 4.89%, or about 100 mg of L-tryptophan.

Magnesium glycinate is a mineral supplement used to prevent and treat low amounts of magnesium in the blood. Magnesium is very important for the normal functioning of cells, nerves, muscles, bones, and the heart. In many embodiments of the present invention, a supplement comprises between about 2.20% to about 26.41%, or between about 45 mg to about 540 mg of magnesium glycinate. In one particular embodiment, the supplement comprises about 8.80%, or about 180 mg of magnesium glycinate.

Melatonin is a naturally produced hormone that helps control sleep and wake cycles. In many embodiments of the present invention, a supplement comprises between about 0.06% to about 0.73%, or between about 1.25 mg to about 15 mg of melatonin. In one particular embodiment, the supplement comprises about 0.24%, or about 5 mg of melatonin.

Niacin, vitamin B3, is required for cell respiration, helps in the release of sleep and metabolism of carbohydrates, fats, and proteins, proper circulation and healthy skin, functioning of the nervous system, and normal secretion of bile and stomach fluids. It is used in the synthesis of sex hormones, treating schizophrenia and other mental illnesses, and a memory-enhancer. Niacin given in pharmaceutical dosage improves the blood cholesterol profile, and has been used to clear the body of organic poisons, such as certain insecticides.

In many embodiments of the present invention, a supplement comprises between about 0.29% to about 3.48%, or between about 8.75 mg to about 105 mg of niacin. In one particular embodiment, the supplement comprises about 1.16%, or about 35 mg of niacin.

Potassium sorbate is the potassium salt of sorbic acid, is used as an additive to inhibit molds and yeasts in many foods, such as cheese, wine, yogurt, dried meats, apple cider, soft drinks and fruit drinks, and baked goods to increase stability and shelf life. In many embodiments of the present invention, a supplement comprises between about 0.25% to about 2.99%, or between about 7.5 mg to about 90 mg of potassium sorbate. In one particular embodiment, the supplement comprises about 1.0%, or about 30 mg of potassium sorbate.

Pyridoxine, also known as vitamin B6, assists in the balancing of sodium and potassium as well as promoting red blood cell production. It is linked to cardiovascular health by decreasing the formation of homocysteine. Pyridoxine may help balance hormonal changes in women and aid the immune system. Most notably, a lack of pyridoxine may cause anemia, nerve damage, seizures, skin problems, and sores in the mouth.

In many embodiments of the present invention, a supplement comprises between about 0.5% to about 4.9%, or between about 10 mg to about 100 mg of pyridoxine. In one particular embodiment, the supplement comprises about 1.47%, or about 30 mg of pyridoxine.

Sucralose is an artificial sweetener that is approximately 600 times as sweet as sucrose (i.e., table sugar), twice as sweet as saccharin, and three times as sweet as aspartame. In many embodiments of the present invention, a supplement comprises between about 1.58% to about 18.91%, or between about 47.5 mg to about 570 mg of sucralose. In one particular embodiment, the supplement comprises about 2.4%, or about 55 mg of sucralose.

Sodium benzoate is a preservative and is most widely used in acidic foods such as salad dressings (e.g., vinegar), carbonated drinks (e.g., carbonic acid), jams and fruit juices (e.g., citric acid), pickles, and condiments. In many embodiments of the present invention, a supplement comprises between about 0.25% to about 2.99%, or between about 7.5 mg to about 90 mg of sodium benzoate. In one particular embodiment, the supplement comprises about 1.0%, or about 30 mg of sodium benzoate.

In accordance with embodiments of the present invention, the inactive ingredients of the supplement may include sweeteners or flavorants. In another embodiment, other inactive ingredients, such as water, may optionally be provided in an amount of between 0.001 ml to about 180 ml, and in one embodiment, about 60 ml.

The supplement can be made in a variety of forms, such as pharmaceutical compositions (e.g., tablet, powder, suspension, liquid, capsule, and gel), nutritional beverages, puddings, confections (e.g., candy), ice cream, frozen confections and/or novelties or non-baked, extruded food products such as bars.

In another embodiment, the ingredients of the supplement can be administered separately, just by incorporating certain components (e.g., bitter tasting ones) into a capsule or tablet and the remaining ingredients provided as a powder or nutritional bar. In one exemplary embodiment, the supplement is provided in a homogenous liquid form. In such an embodiment, the supplement may be packaged in a single dose bottle, e.g., a shot-sized bottle about 50 ml to about 75 ml in volume. The supplement can be formulated for single or multiple daily administration.

While the foregoing is directed to embodiments of the present invention, other and further embodiments of the invention may be devised without departing from the basic scope thereof. For example, although numerous embodiments having various features have been described herein, combinations of such various features in other combinations not discussed herein are contemplated within the scope of embodiments of the present invention.

What is claimed is:

1. A nutritional sleep supplement having a set of active ingredients, the active ingredients comprising:
between about 50 mg to about 600 mg of 5-Hydroxytryptophan;
between about 125 mg to about 1500 mg of calcium lactate;
between about 50 mg to about 600 mg of citric acid;
between about 125 mg to about 1500 mg of gamma-Aminobutyric acid;
between about 50 mg to about 600 mg of L-theanine;
between about 25 mg to about 300 mg of L-tryptophan;
between about 45 mg to about 540 mg of magnesium glycinate;
between about 1.25 mg to about 15 mg of melatonin;
between about 8.75 mg to about 105 mg of niacin;
between about 7.5 mg to about 90 mg of potassium sorbate; and
between about 10 mg to about 100 mg of pyridoxine.

2. The nutritional sleep supplement of claim 1, further having a set of inactive ingredients, the inactive ingredients comprising:
between about 47.5 mg to about 570 mg of sucralose;
between about 7.5 mg to about 90 mg of sodium benzoate; and
between about 0.001 ml to about 180 ml of water.

3. The nutritional sleep supplement of claim 1, wherein the supplement is prepared as one of a tablet, a powder, a suspension, a liquid, a capsule, a gel, a beverage, a pudding, a confection, an ice cream, a frozen confection or a non-baked, extruded food bar.

4. The nutritional sleep supplement of claim 3, wherein the supplement is prepared in liquid form, and packaged in at least about a 50 ml to about a 75 ml bottle.

5. The nutritional sleep supplement of claim 1, wherein the active ingredients comprise:
about 200 mg of 5-Hydroxytryptophan;
about 500 mg of calcium lactate;
about 200 mg of citric acid;
about 750 mg of gamma-Aminobutyric acid;
about 200 mg of L-theanine;
about 100 mg of L-tryptophan;
about 180 mg of magnesium glycinate;
about 5 mg of melatonin;
about 15 mg of niacin;
about 30 mg of potassium sorbate; and
about 30 mg of pyridoxine.

6. The nutritional sleep supplement of claim 5, further having a set of inactive ingredients, the inactive ingredients comprising:
about 55 mg of sucralose;
about 30 mg of sodium benzoate; and
about 60 ml of water.

7. A nutritional sleep supplement comprising:
between about 50 mg to about 600 mg of 5-Hydroxytryptophan;
between about 125 mg to about 1500 mg of calcium lactate;
between about 50 mg to about 600 mg of citric acid;
between about 125 mg to about 1500 mg of gamma-Aminobutyric acid;
between about 50 mg to about 600 mg of L-theanine;
between about 25 mg to about 300 mg of L-tryptophan;
between about 45 mg to about 540 mg of magnesium glycinate;
between about 1.25 mg to about 15 mg of melatonin;
between about 8.75 mg to about 105 mg of niacin;
between about 7.5 mg to about 90 mg of potassium sorbate
between about 10 mg to about 100 mg of pyridoxine;
between about 47.5 mg to about 570 mg of sucralose;
between about 7.5 mg to about 90 mg of sodium benzoate; and
between about 0.001 ml to about 180 ml of water.

8. The nutritional sleep supplement of claim 7, wherein the supplement comprises:
about 200 mg of 5-Hydroxytryptophan;
about 500 mg of calcium lactate;
about 200 mg of citric acid;
about 750 mg of gamma-Aminobutyric acid;
about 200 mg of L-theanine;
about 100 mg of L-tryptophan;
about 180 mg of magnesium glycinate;
about 5 mg of melatonin;
about 15 mg of niacin;
about 30 mg of potassium sorbate;
about 30 mg of pyridoxine;
about 55 mg of sucralose;
about 30 mg of sodium benzoate; and
about 60 ml of water.

9. A nutritional sleep supplement consisting of:
between about 50 mg to about 600 mg of 5-Hydroxytryptophan;
between about 125 mg to about 1500 mg of calcium lactate;
between about 50 mg to about 600 mg of citric acid;
between about 125 mg to about 1500 mg of gamma-Aminobutyric acid;
between about 50 mg to about 600 mg of L-theanine;
between about 25 mg to about 300 mg of L-tryptophan;
between about 45 mg to about 540 mg of magnesium glycinate;
between about 1.25 mg to about 15 mg of melatonin;
between about 8.75 mg to about 105 mg of niacin;
between about 7.5 mg to about 90 mg of potassium sorbate
between about 10 mg to about 100 mg of pyridoxine;
between about 47.5 mg to about 570 mg of sucralose;
between about 7.5 mg to about 90 mg of sodium benzoate; and
between about 0.001 ml to about 180 ml of water.

10. The nutritional sleep supplement of claim 9, wherein the supplement consists of:
about 200 mg of 5-Hydroxytryptophan;
about 500 mg of calcium lactate;
about 200 mg of citric acid;
about 750 mg of gamma-Aminobutyric acid;
about 200 mg of L-theanine;
about 100 mg of L-tryptophan;
about 180 mg of magnesium glycinate;
about 5 mg of melatonin;
about 15 mg of niacin;
about 30 mg of potassium sorbate;
about 30 mg of pyridoxine;
about 55 mg of sucralose;
about 30 mg of sodium benzoate; and
about 60 ml of water.
